(19) United States
(12) Patent Application Publication
(10) Pub. No.: US 2015/0366613 A1
(43) Pub. Date: Dec. 24, 2015

(54) ABLATION PROBE WITH METALIZED CERAMIC COMPONENT

(71) Applicant: Perseon Corporation, Salt Lake City, UT (US)

(72) Inventors: Chet M. Crump, South Jordan, UT (US); Kent Moore, Bountiful, UT (US); Mark Sedlacek, Salt Lake City, UT (US); Todd H. Turnlund, Park City, UT (US)

(21) Appl. No.: 14/310,975

(22) Filed: Jun. 20, 2014

(51) Int. Cl. A61B 18/18

(52) U.S. Cl. CPC A61B 18/1815; A61B 2018/00077

(57) ABSTRACT

Ablation probes can include one or more metalized ceramic components. A metalized ceramic component can include one or more traces for conducting electrical energy and/or for functioning as an antenna for emitting radiation during an ablation procedure. A shaft of an ablation probe may be formed of metalized ceramic to give the shaft strength and to provide an electrical insulator between traces formed on the shaft and other components of the probe. A tip of an ablation probe may also be formed of metalized ceramic.

### CROSS-REFERENCE TO RELATED APPLICATIONS

N/A

### BACKGROUND

Microwave ablation (MWA) is a medical procedure where in vivo tissue is ablated using high frequency electromagnetic field to treat a medical disorder. MWA is commonly performed to treat tumors in body organs. During MWA, a needle-like MWA probe is placed inside the tumor. Microwaves emitted from the probe heat surrounding tumor tissue, destroying the target tissues, such as soft tissue, cancerous tumor, nerve, or other target structure. Cancer cells, in particular, break down and die at elevated temperatures caused by MWA procedures. Some MWA procedures create temperatures up to or exceeding 300 degrees Celsius.

For MWA to be successful, a sufficient amount of molecular agitation must occur within the tissue. For example, the varying electromagnetic field generated by the waves emitted from the MWA probe causes water molecules to rapidly vibrate as they attempt to align with the varying field. This molecular agitation creates frictional heat which is capable of rapidly increasing the temperature of the tissue in a similar manner as a microwave oven heats food.

It is desirable to heat the entire area of the tumor with a single treatment. However, it is difficult to obtain even heat distribution using current ablation techniques. When heated to above 60° C., tissue will immediately coagulate.

### BRIEF SUMMARY

The present invention extends to ablation probes that include one or more metalized ceramic components. A metalized ceramic component can include one or more traces for conducting electrical energy and/or for functioning as an antenna for emitting radiation during an ablation procedure. A shaft of an ablation probe may be formed of metalized ceramic to give the shaft strength and to provide an electrical insulator between traces formed on the shaft and other components of the probe. A tip of an ablation probe may also be formed of metalized ceramic.

In one embodiment, the present invention is implemented as an ablation probe that comprises a shaft formed of a metalized ceramic. The shaft may be metalized by forming one or more traces along a surface of the shaft. The one or more traces may extend along an outer and/or inner surface of the shaft.

The one or more traces may extend along a surface of the shaft that is in contact with a tip or a proximal shaft of the ablation probe. The tip or proximal shaft may be formed of various materials. Non-limiting examples of different tip materials include a metalized ceramic, a conductive material, a non-conductive, low loss dielectric insulator with low dielectric constant (insulator), PVC, fiberglass, PEEK, nylon, etc. The tip or proximal shaft may connect the one or more traces to a conductor that extends within the shaft. A tip formed of metalized ceramic may include a tip trace that contacts at least one of the one or more traces on the shaft.

The ablation probe may also comprise one or more coatings on the surface of the shaft covering at least a portion of the one or more traces. The one or more traces may function as an antenna for the ablation probe which transmits electromagnetic waves. The one or more traces may comprise a distal trace and a proximal trace. The distal trace may be electrically connected to a first conductor and the proximal trace may be electrically connected to a second conductor. The distal trace may also be electrically connected to the proximal trace. At least one of the one or more traces may have a varied dimension or pattern.

In another embodiment, the present invention is implemented as an ablation probe that comprises a shaft that is formed of ceramic and that includes one or more metal traces formed on a surface of the shaft, and a tip configured for insertion into a patient to perform an ablation procedure. The tip may be formed of a conductive material, and at least one of the one or more metal traces may be in contact with the tip for receiving electrical energy that is conducted through the tip. At least one of the one or more metal traces may be formed on an outer surface of the shaft. At least one of the one or more metal traces may have a varied dimension or pattern. The tip may also be formed of ceramic. The shaft and the tip may comprise a single component. The tip may include one or more metal traces on a surface of the tip that are connected to the one or more metal traces on the surface of the shaft.

In another embodiment, the present invention is implemented as an ablation probe that comprises a body that is formed of ceramic, and one or more traces formed on a surface of the body. The one or more traces may form an antenna. The body may include a tip. The one or more traces may be formed on one or both of an outer surface or an inner surface of the body. At least one of the one or more traces may have a varied dimension or pattern.

This summary is provided to introduce a selection of concepts in a simplified form that are further described below in the Detailed Description. This Summary is not intended to identify key features or essential features of the claimed subject matter.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features of the invention can be obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings.

FIG. 1 illustrates an ablation emitter assembly that includes an ablation probe in accordance with one or more embodiments of the present invention.

FIG. 2 illustrates a portion of an ablation probe that includes one or more metalized ceramic components.

FIG. 3 illustrates a portion of another ablation probe that includes one or more metalized ceramic components.

FIG. 3A provides a cross-sectional view of the ablation probe of FIG. 3 in which the shaft is formed of metalized ceramic.

FIG. 3B provides a cross-sectional view of the ablation probe of FIG. 3 in which the shaft and the tip are formed of metalized ceramic.

FIG. 3C provides a cross-sectional view of the ablation probe of FIG. 3 in which the conductor is connected directly a portion of the trace that is formed on an inner surface of the shaft.

FIG. 4 illustrates a portion of another ablation probe that includes one or more metalized ceramic components.

FIG. 4A provides a cross-sectional view of the ablation probe of FIG. 4 in which the shaft is formed of metalized ceramic.

FIG. 4B provides a cross-sectional view of the ablation probe of FIG. 4 in which the shaft and the proximal shaft are formed of metalized ceramic.

FIG. 4C provides a cross-sectional view of the ablation probe of FIG. 4 in which the conductor is connected directly a portion of the trace that is formed on an inner surface of the shaft.

FIG. 5 illustrates a portion of another ablation probe that includes one or more metalized ceramic components.

FIG. 5A provides a cross-sectional view of the ablation probe of FIG. 5 in which the shaft is formed of metalized ceramic.

FIG. 5B provides a cross-sectional view of the ablation probe of FIG. 5 in which the shaft, the distal shaft, and the tip are formed of metalized ceramic.

FIG. 6 provides a cross-sectional view of another ablation probe that includes a shaft formed of metalized ceramic and an internal antenna element.

FIG. 7 illustrates an embodiment of a probe having a trace with a varying pitch.

FIG. 8 illustrates an embodiment of a probe having a trace with a varying width.

FIG. 9 illustrates an embodiment of a probe having multiple traces that extend in a proximal and a distal direction.

FIG. 10 illustrates a partially transparent front view of an embodiment of a probe that includes one or more ceramic components.

FIGS. 11A-11C illustrate different views of a probe that is comprised of ceramic.

FIG. 12 illustrates a portion of another ablation probe that includes one or more metalized ceramic components.

FIG. 13 illustrates a portion of another ablation probe that includes one or more metalized ceramic components.

### DETAILED DESCRIPTION

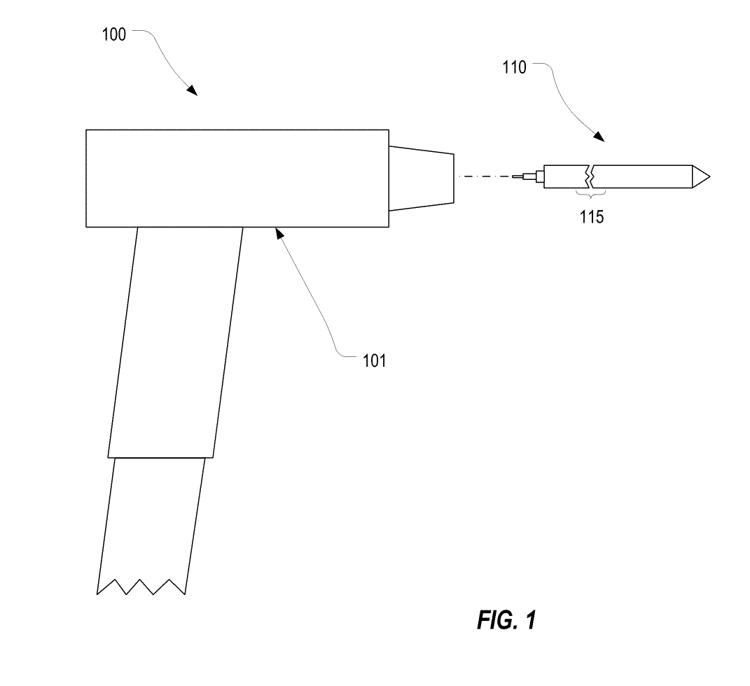

FIG. 1 is intended to provide an overview of the general architecture of a microwave ablation (MWA) device 100 that can be used in MWA procedures. The MWA device 100 can include a body 101 and a probe 110 that is configured to attach to and extend from a distal end of body 101. Probe 110 can have various lengths as indicated by the break 115 in FIG. 1 and may typically be between 1 and 12 inches. The gauge of probe 110 can range between 8 to 24, including, but not limited to, an 11, 13, 14, 16, 17, or 18 gauge.

Body 101 typically includes (or provides access to) a microwave power source (not shown) for supplying microwave energy to probe 110. Probe 110 comprises an antenna for emitting the microwave energy into surrounding tissue when probe 110 is inserted within a patient's tissue.

Body 101 may also include (or provide access to) a controller (not shown) for controlling the power, frequency, and/or phase of the microwave energy delivered to probe 110. In some embodiments, the controller can be configured to automatically adjust the power, frequency, and/or phase of the microwave energy delivered to probe 110 in order to tune or impedance match the probe to surrounding tissue.

The MWA device 100 can be configured to transmit energy having one or more frequencies or a variable frequency. For example, in some embodiments, the microwave power source is a microwave source configured to provide microwave energy to probe 110. Such energy can have a frequency within the range of about 300 MHz to 30 GHz. In some embodiments, a specific frequency of 915 or 2,450 MHz may be preferred. When microwave energy is delivered to probe 110, tissue surrounding probe 110 can be ablated by heat generated by probe 110.

Additionally, the microwave power source can be configured to transmit various levels of energy to probe 110. In some embodiments, the microwave power source can transmit up to about 300 W of power to probe 110. In other embodiments, the microwave power source can transmit between 0 W to 300 W of power to probe 110, including specifically transmitting up to 40 W, up to 60 W, up to 120 W, up to 180 W, or up to 240 W of power to probe 110.

In some embodiments, the controller can be configured to ramp up the power delivered to probe 110 slowly during the initial phases of an ablation procedure. Such configurations can incrementally, exponentially, or otherwise ramp up power from zero to a maximum power output over a predetermined time. For instance, the controller can be configured to ramp up power delivered to probe 110 from 0 W to 60 W over a time period.

During MWA, probe 110 is inserted through the skin and tissue of a patient, and is then directed toward a target structure, such as a tumor, cell(s), or nerve(s). Probe 110 can be inserted into the target structure or placed beside the target structure. Microwave energy emitted from probe 110 can then heat the target structure, which may be ablated and/or killed. When the target structure is exposed to the transmitted microwave energy for an adequate amount of time and temperature, the target structure can be ablated. Cancer cells, in particular, can break down and die at elevated temperatures caused by MWA ablation procedures. Some MWA procedures create temperatures up to or exceeding 100 to 350 degrees Celsius.

Generally, the shape and size of an ablation pattern produced by probe 110 roughly corresponds to the shape and intensity of the microwave transmission patterns of the waves emitted from probe 110. Thus, a substantially spherical transmission pattern can produce a roughly spherical ablation pattern. Accordingly, probe 110 can be configured to produce ablation regions that are substantially the same size as the target structure so that the appropriate amount of target tissue is ablated, without ablating healthy surrounding tissues. For example, since many tumors are approximately spherical, probe 110 can be configured to produce a generally spherical ablation region.

Additionally, probe 110 can be configured to produce ablation regions that are directional and dose-able (or shapeable) so that they can be shaped to be the same size as a target structure or so that they can be directed toward a target structure near probe 110. Such directionality can be produced, in some instances, by varying the phase between transmitted microwave energy transmitted through multiple conductors of probe 110.

In accordance with embodiments of the present invention, one or more components of an ablation probe (e.g. probe 110) can be formed of metalized ceramic. Metallizing ceramic refers to the process of applying one or more layers of metal on the surface of the ceramic and then heating the ceramic to cause the metal to bond with the ceramic. Various techniques exist for metallizing ceramic that would be suitable for metallizing a component of an emitter assembly. For example, a thick film ink containing a moly manganese refractory formula or another metal can be applied through a screen, roll printing, hand painting, air brush spraying, immersion, centrifugal coating, needle painting, etc. to a ceramic component and fired at temperatures sufficient to cause bonding of the metal to the ceramic.

A metalized ceramic is therefore a ceramic material having metal applied and heated on its surface to fuse the metal to the surface. For example, metal traces can be placed or deposited on the surface of the ceramic to form an antenna or other electrical component or connector of an ablation probe. Suitable ceramics that can be metalized include aluminum oxide, zirconia toughened alumina, zirconia, partially stabilized zirconia, aluminum nitride, silicon carbide, or other ceramic material. Many different types of metal can be used to metalize ceramics including silver, copper, gold, aluminum, nickel, molybdenum (“moly”) manganese, brass, or other conductive elements or alloyed elements.

Metalized ceramics provide strong adherence of metal to ceramic, excellent electrical and mechanical properties, high electrical conductivity, hermetic sealing capability, flexible three-dimensional designs, and adaptation to metal/ceramic components/assemblies. Aluminum oxide ceramic can be preferred in some embodiments because it is an electrical insulator that is also strong and tolerant of high temperatures. Aluminum oxide also has a moderate thermal conductivity. Accordingly, a component formed of an aluminum oxide ceramic can provide high electrical insulation between different components of a probe while also providing thermal conductance to allow heat to be dissipated. Other ceramic materials may also provide similar benefits.

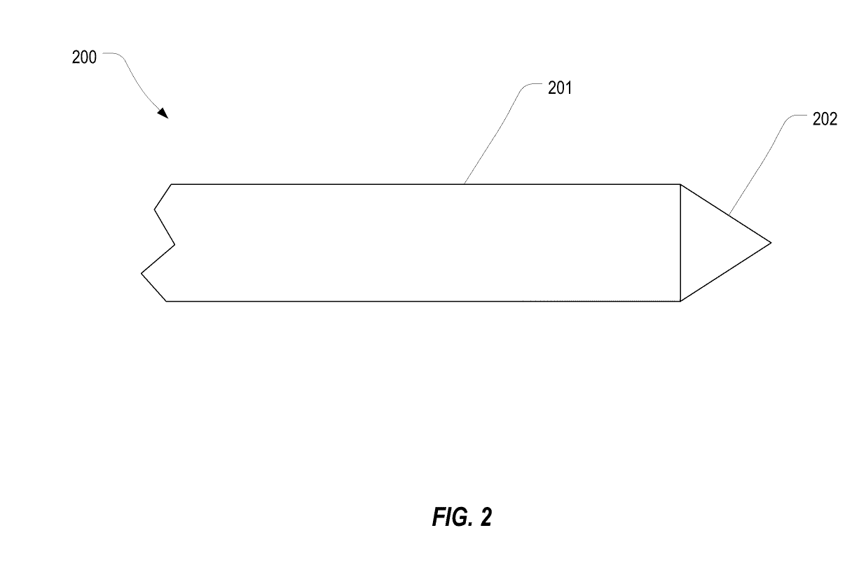

FIG. 2 illustrates a portion of an ablation probe 200. Ablation probe 200 includes a shaft 201 and a tip 202 which may be separate components or the same component. In accordance with embodiments of the present invention, shaft 201 and/or tip 202 can be formed of ceramic such as aluminum oxide. Shaft 201 and/or tip 202 can also be metalized in that metal can be applied on at least a portion of the surface of the component. For example, metal traces can be placed or deposited on an inner and/or outer surface of shaft 201 and/or tip 202.

In typical ablation probe configurations, a conductor extends through the interior of the probe to carry electrical energy to an antenna formed at or near a distal end of the probe. In some embodiments, metal traces formed on an inner and/or outer surface of shaft 201 and/or tip 202 can be connected to this conductor and function as an antenna for emitting electromagnetic waves from the probe.

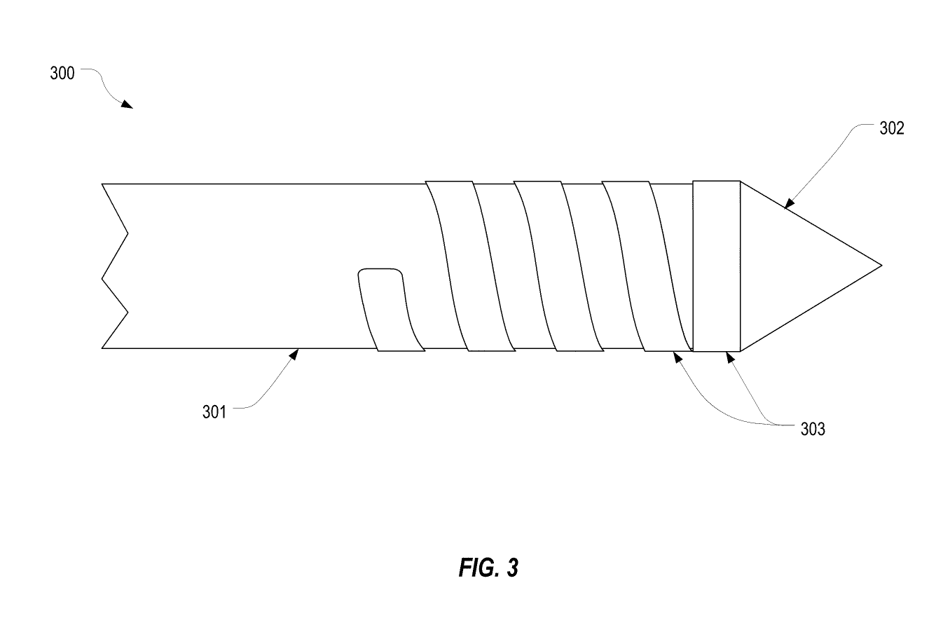

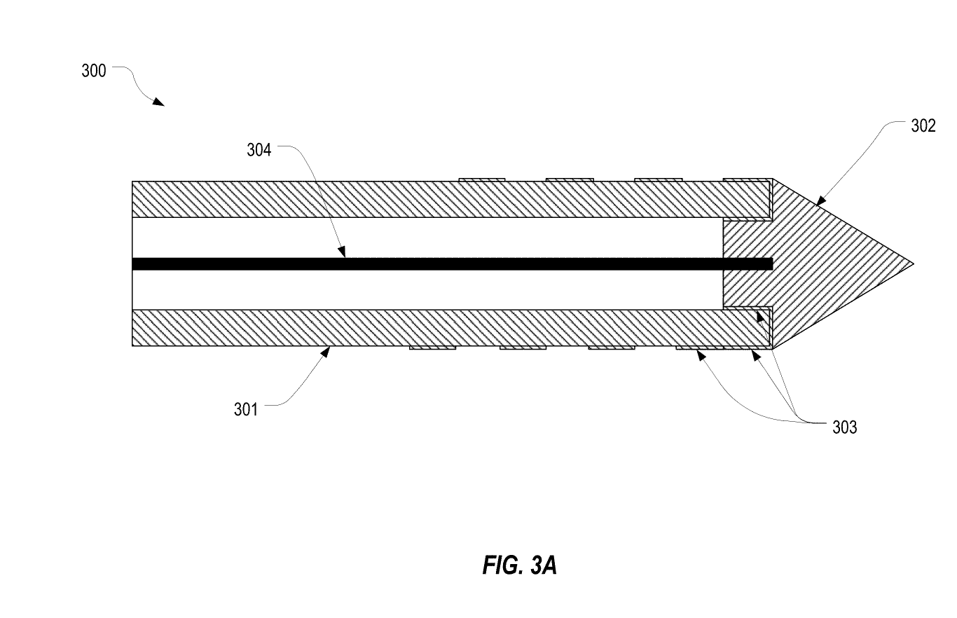

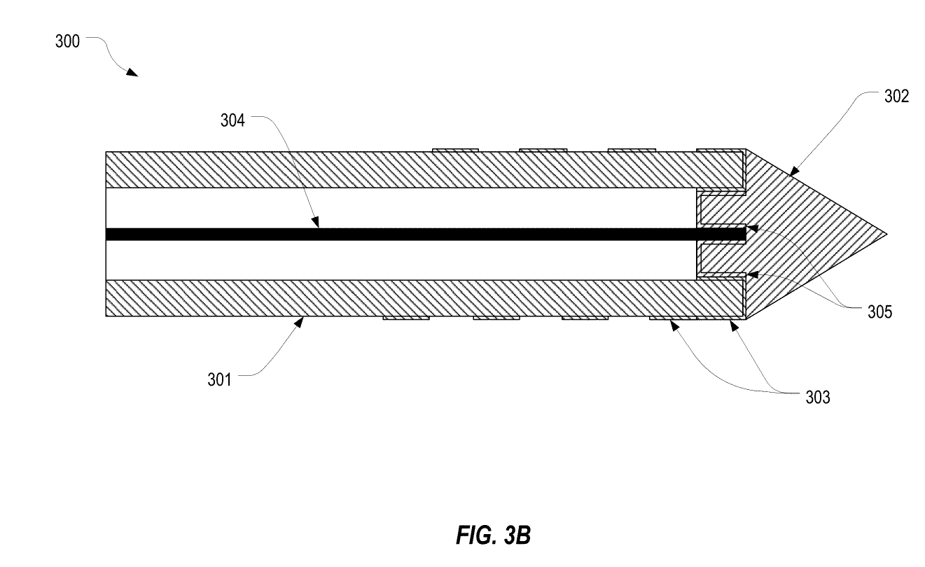

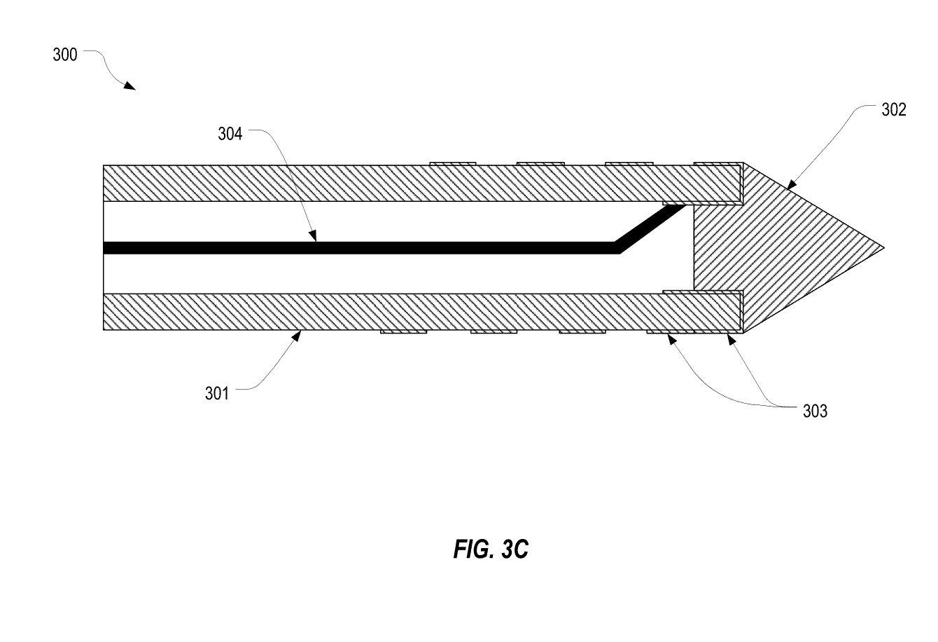

FIGS. 3 and 3A-3C illustrate an embodiment where an ablation probe 300 includes a metalized ceramic shaft 301. A trace 303 is formed on an outer surface of shaft 301 and functions as an antenna for emitting electrical energy supplied by a conductor 304. Trace 303 extends in a proximal direction away from tip 302. Trace 303 can be connected to an inner conductor for receiving electrical energy in various ways including via tip 302 or a direct connection to the inner conductor.

FIG. 3A provides a cross-sectional view of probe 300 in an embodiment where tip 302 is formed of a conductive material, such as brass, titanium, or another metal, which connects trace 303 to conductor 304. As shown, trace 303 can extend around a distal end of shaft 301 onto an inner surface of shaft 301 so that trace 303 is in contact with tip 302. Conductor 304 can also extend into tip 302. Therefore, electrical energy carried by conductor 304 will be conducted through tip 302 to trace 303. In some embodiments, trace 303 may only extend around the distal end but may not extend onto the inner surface of shaft 301.

FIG. 3B provides a cross-sectional view of probe 300 in an embodiment where tip 302 is formed of a non-conductive material such as ceramic. In this embodiment, tip 302 can be metalized by forming a trace 305 along the surfaces shown in FIG. 3B. Trace 305 can contact conductor 304 and trace 303 thereby allowing electrical energy to flow from conductor 304 to trace 303.

FIG. 3C provides a cross-sectional view of probe 300 in an embodiment where conductor 304 connects directly to a portion of trace 303 that extends along an inner surface of shaft 301. In this embodiment, tip 302 may be formed of a non-conductive material, although it may also equally be formed of a conductive material. Also, in some embodiments, tip 302 and shaft 301 may be a unitary component. In such embodiments, trace 303 may extend through a hole or other channel within shaft 301/tip 302 to form a surface to which conductor 304 may connect. In some embodiments, conductor 304 may also be connected to both tip 302 and trace 303.

In addition to the helical pattern of trace 303 shown in FIG. 3, any other pattern could be used for trace 303. For example, a zigzag or stepped pattern could be employed. Trace 303 may also include one or more extensions that divert from the pattern. For example, a portion of trace 303 may extend proximally from a proximal end of the helical pattern. Similarly, more than one trace may extend from the distal end of shaft 301. Accordingly, the present invention should extend to embodiments that include traces of any pattern, orientation, shape, dimension, etc.

FIGS. 3 and 3A-3C illustrate one example where a trace extends from a distal end of shaft 301. The present invention also encompasses embodiments where one or more traces extend only from a proximal end of shaft 301, or one or more traces extend from both a proximal and distal end of shaft 301 including when these traces are connected. Some examples of these various configurations are further described below.

In some embodiments, trace 303 may have a variable pattern. For example, the pitch of a helical or other pattern may vary. Varying the pitch can change the field intensity of the microwaves emitted from the trace. A smaller pitch will cause the windings of a helical trace to be spaced more closely and will therefore increase the field intensity along the portion of the emitter assembly with the smaller pitched trace. Traces that are more closely spaced will also create a greater density of heat. Accordingly, the pitch of a trace may be reduced nearer the proximal end of shaft 301 so that the heat density is greatest nearer a proximal end where the heat may be more readily dissipated. In some embodiments, proximal and distal portions of trace 303 may have a smaller pitch than a middle portion of the trace such that the trace is more closely spaced in the proximal and distal portions than in the middle portion. Varying the pitch in this manner can create a spherical ablation pattern.

The width of trace 303 may also be varied. A thicker trace will allow more current flow. Accordingly, in some embodiments, a distal portion of trace 303 may be thicker than a proximal portion to account for higher currents that pass through the distal portion.

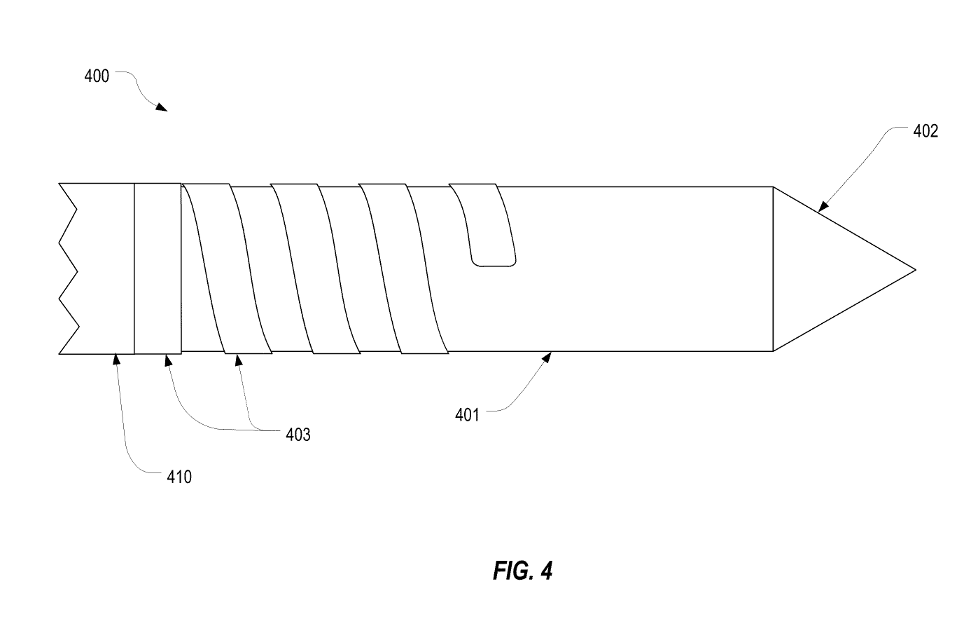

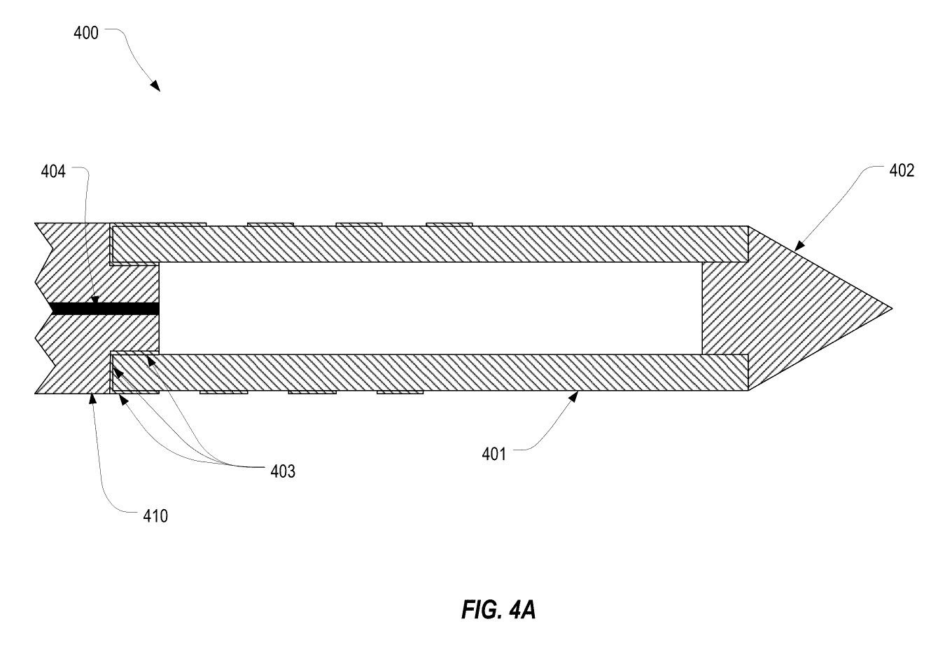

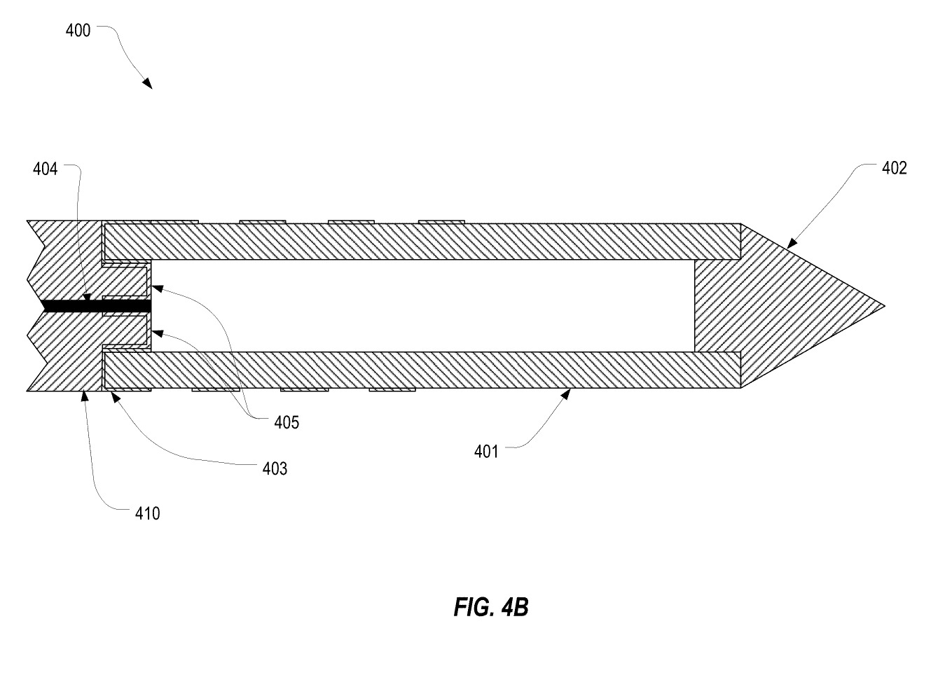

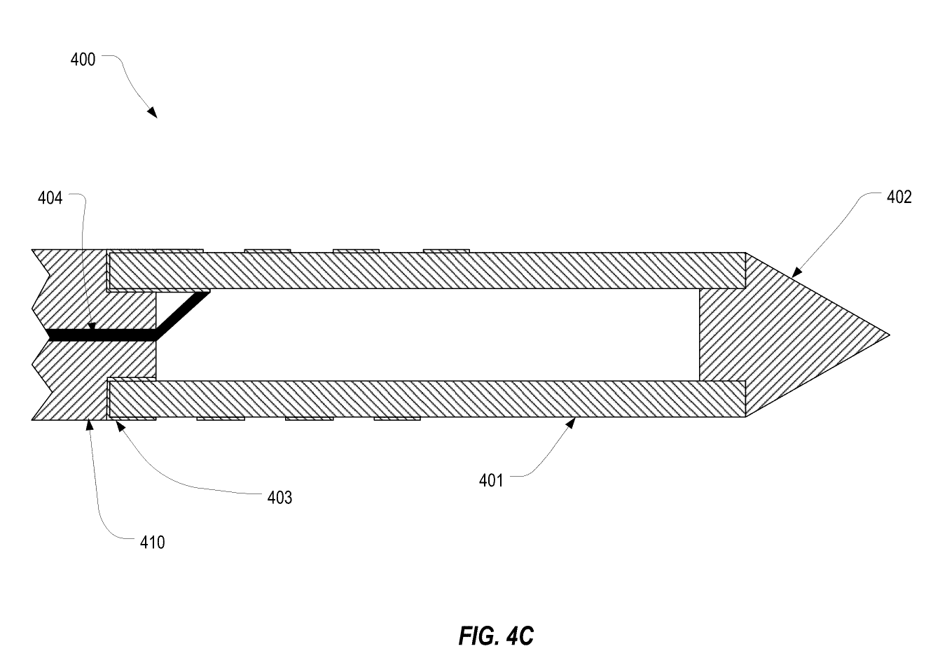

FIGS. 4 and 4A-4C illustrate another embodiment where an ablation probe 400 includes a metalized ceramic shaft 401. In contrast to probe 300, probe 400 includes a trace 403 that extends in a distal direction towards tip 402. As with trace 303, trace 403 can be connected to an inner conductor for receiving electrical energy in various ways including via proximal shaft 410 or a direct connection to the inner conductor. Trace 403 can also have any suitable pattern, shape, dimension, etc. beyond what is shown in FIG. 4 including a variable pattern, shape, or dimension as described above.

Although FIG. 4 depicts tip 402 as being a separate component from shaft 401, in some embodiments, shaft 401 and tip 402 may comprise a single unitary component. For example, shaft 401/tip 402 could be a single ceramic component. Similarly, in some embodiments, shaft 401 (or equally another shaft described herein) may be configured to connect to a two piece or other multiple piece tip.

FIG. 4A provides a cross-sectional view of probe 400 in an embodiment where proximal shaft 410 is formed of a conductive material, such as brass, titanium, or another metal, which connects trace 403 to conductor 404. As shown, trace 403 can extend around a proximal end of shaft 401 onto an inner surface of shaft 401 so that trace 403 is in contact with proximal shaft 410. Conductor 404 can also extend into proximal shaft 410. Therefore, electrical energy carried by conductor 404 will be conducted through proximal shaft 410 to trace 403. In some embodiments, trace 403 may only extend around the proximal end but may not extend onto the inner surface of shaft 401.

FIG. 4B provides a cross-sectional view of probe 400 in an embodiment where proximal shaft 410 is formed of a non-conductive material such as ceramic. In this embodiment, proximal shaft 410 can be metalized by forming a trace 405 along the surfaces shown in FIG. 4B. Trace 405 can contact conductor 404 and trace 403 thereby allowing electrical energy to flow from conductor 404 to trace 403.

FIG. 4C provides a cross-sectional view of probe 400 in an embodiment where conductor 404 connects directly to a portion of trace 403 that extends along an inner surface of shaft 401. In this embodiment, proximal shaft 410 may typically be formed of a non-conductive material, although it may also equally be formed of a conductive material. In some embodiments (not shown), a separate proximal shaft may not be used. In such embodiments, trace 403 may extend through a hole or other channel within shaft 401 to form a surface to which conductor 404 may connect.

In FIGS. 4A-4C, proximal shaft 410 is shown as inserting into shaft 401. However, proximal shaft 410 may also be configured to abut shaft 401 rather than insert into shaft 401 or may both abut shaft 401 and insert into shaft 401. Also, proximal shaft 410 may be configured with a similar internal diameter as shaft 401 where they can be connected to each other or via a coupling tube.

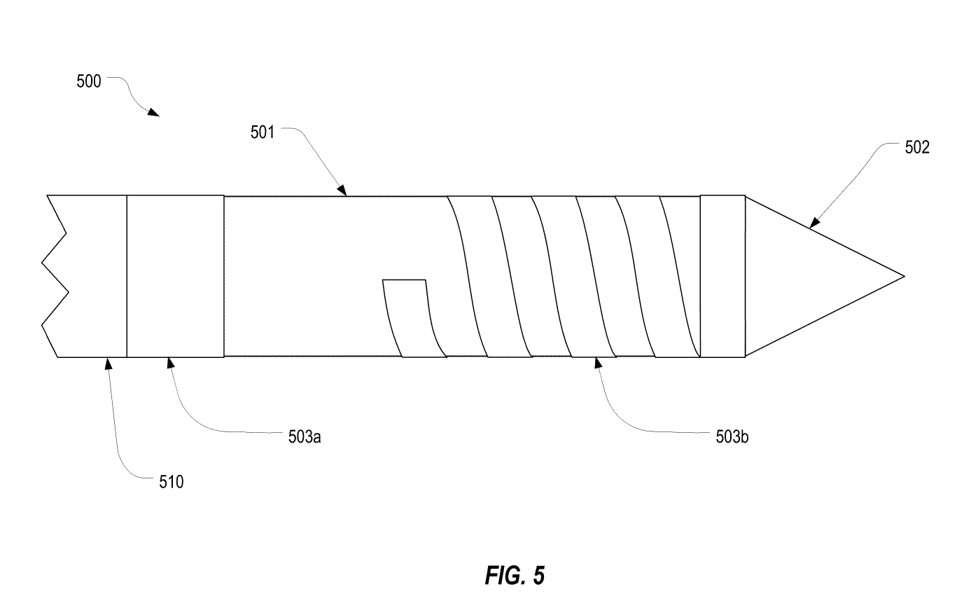

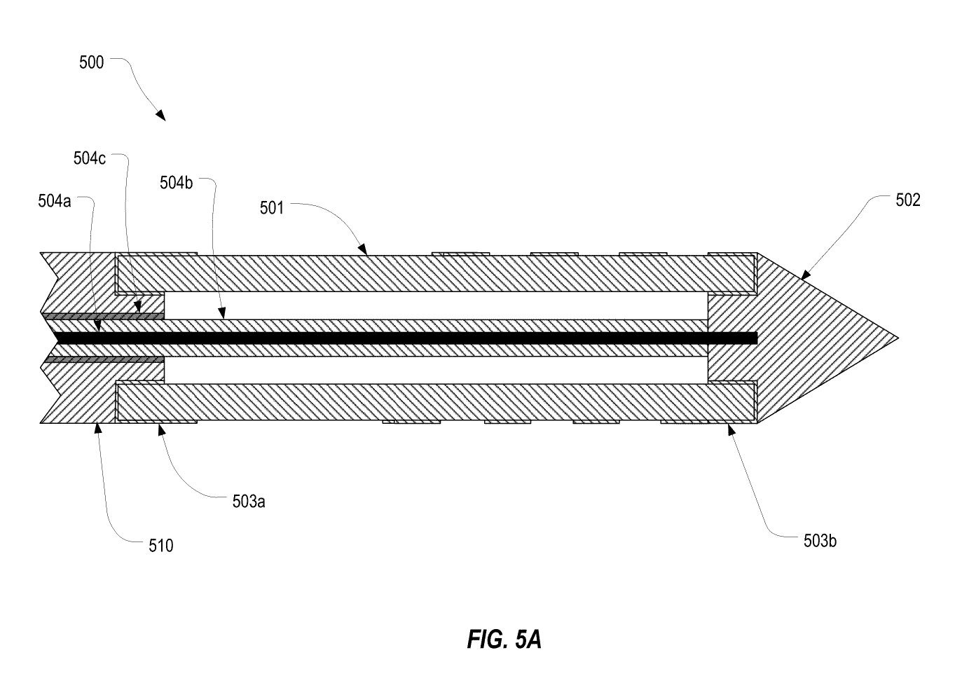

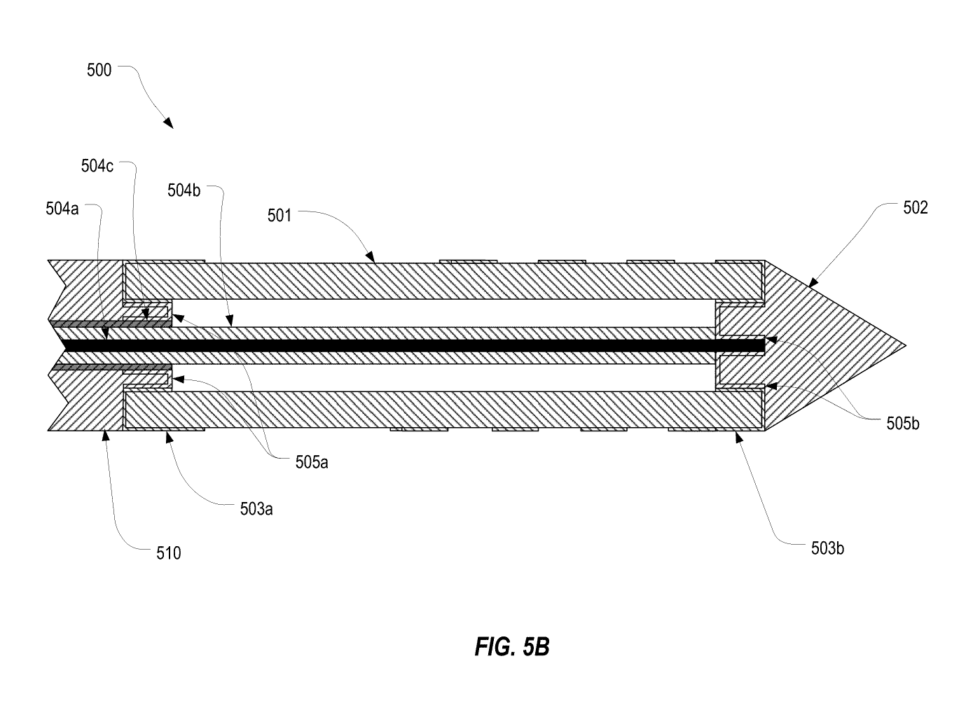

FIGS. 5, 5A, and 5B illustrate another embodiment where an ablation probe 500 includes a metalized ceramic shaft 501. Probe 500 is similar to probe 300 but includes two separate traces 503a and 503b on its outer surface. Trace 503a extends in a distal direction towards tip 502 while trace 503b extends in a proximal direction away from tip 502. Each of traces 503a and 503b can be connected to a conductor in various ways including via proximal shaft 510 or tip 502 respectively or via a direct connection. In some embodiments, trace 503a may be configured in a similar manner as trace 503b (i.e. trace 503a may have a helical shape that extends distally towards trace 503b). In some embodiments, a gap may remain between trace 503a and trace 503b, while in others, trace 503a and trace 503b may connect. As with traces 303 and 403, traces 503a and 503b can have any suitable pattern, shape, dimension, etc. including a variable pattern, shape, or dimension as described above.

FIG. 5A provides a cross-sectional view of probe 500 in an embodiment where proximal shaft 510 and tip 502 are formed of a conductive material, such as brass, titanium, or another metal. Trace 503a can extend around a proximal end of shaft 501 onto an inner surface of shaft 501 so that trace 503a is in contact with proximal shaft 510. Similarly, trace 503b can extend around a distal end of shaft 501 onto an inner surface of shaft 501 so that trace 503b is in contact with tip 502. An inner conductor 504a may extend into tip 502 while an outer conductor 504c, which is separated from inner conductor 504a by an insulator 504b, may be in contact with proximal shaft 510. Therefore, trace 503a is electrically connected to outer conductor 504c and trace 503b is electrically connected to inner conductor 504a.

FIG. 5B provides a cross-sectional view of probe 500 in an embodiment where proximal shaft 510 and tip 502 are formed of a non-conductive material such as ceramic. In this embodiment, proximal shaft 510 and tip 502 can be metalized by forming traces 505a and 505b along the surfaces shown in FIG. 5B. Trace 505a can contact outer conductor 504c and trace 503a thereby allowing electrical energy to flow between outer conductor 504c and trace 503a. Similarly, trace 505b can contact inner conductor 504a and trace 503b thereby allowing electrical energy to flow between inner conductor 504a and trace 503b.

Probe 500 may also be configured in a similar manner as is shown in FIG. 3C. In particular, inner conductor 504a may be configured to directly connect to a portion of trace 503b that extends along an inner surface of shaft 501. In such embodiments, tip 502 may typically be formed of a non-conductive material, although it may also equally be formed of a conductive material. Also, in some embodiments, tip 502 and shaft 501 may be a unitary component. In such embodiments, trace 503b may extend through a hole or other channel within shaft 501/tip 502 to form a surface to which inner conductor 504a may connect.

In addition to the embodiments shown in FIGS. 5A and 5B, only one of proximal shaft 510 or tip 502 may be formed of a non-conductive material. Also, traces 503a and 503b may be configured in different patterns, orientations, shapes, dimensions, etc. For example, trace 503a may extend a farther distance along shaft 501 while trace 503b may extend a shorter distance than what is shown in these figures. Also, in some embodiments, traces 503a and 503b may be connected. For example, the helical pattern of trace 503b may continue up to trace 503a. Alternatively, an extension may be formed that connects the helical portion of trace 503b to trace 503a.

Probes 300, 400, and 500 are all depicted as having a conically-shaped tip. However, any shaped tip may equally be used. Also, although FIGS. 3B, 4B, and 5B depict that traces 305, 405, and 505b extend around a proximal end of tip 302, 402, and 502 respectively, in some embodiments, traces 305, 405, and 505b may alternatively extend through (or otherwise be formed within) a channel that extends through the tip. For example, the channel into which conductor 304, 404, and 504a inserts may extend to an outer surface of the tip. In such cases, traces 305, 405, or 505b may be formed on a surface of this channel and extend along the outer surface of the tip until contacting trace 303, 403, or 503b respectively. Similar configurations could be used on proximal shaft 310, 410, or 510. Accordingly, the present invention encompasses metalized tips or proximal shafts regardless of how the tip or proximal shaft is metalized.

The present invention also encompasses embodiments where a trace extends in a distal direction on an outer surface of the tip. For example, referring to FIG. 3B, trace 303 (or trace 305) may extend distally along tip 302. The present invention also encompasses embodiments where a trace extends only on an outer surface of the tip. For example, referring again to FIG. 3B, trace 305 can be configured to extend to an outer surface of tip 302 to form an antenna on the tip while trace 303 is not included on shaft 301.

Additionally, although the above description has generally treated the tip and the shaft as separate components, the present invention encompasses embodiments where the tip and the shaft are the same component. For example, the shaft and tip portions shown in the figures could be formed of a single piece of ceramic that is metalized with one or more traces. These traces may extend distally towards the tip such as is shown in FIGS. 4A and 4B, or may extend out through a channel in the tip and then extend proximally away from the tip.

Further, the present invention encompasses embodiments where the traces are formed only on an inner surface of a metalized ceramic component. For example, as opposed to being formed on an outer surface as shown in the figures, traces 303, 403, 503a, or 503b can be formed on an inner surface.

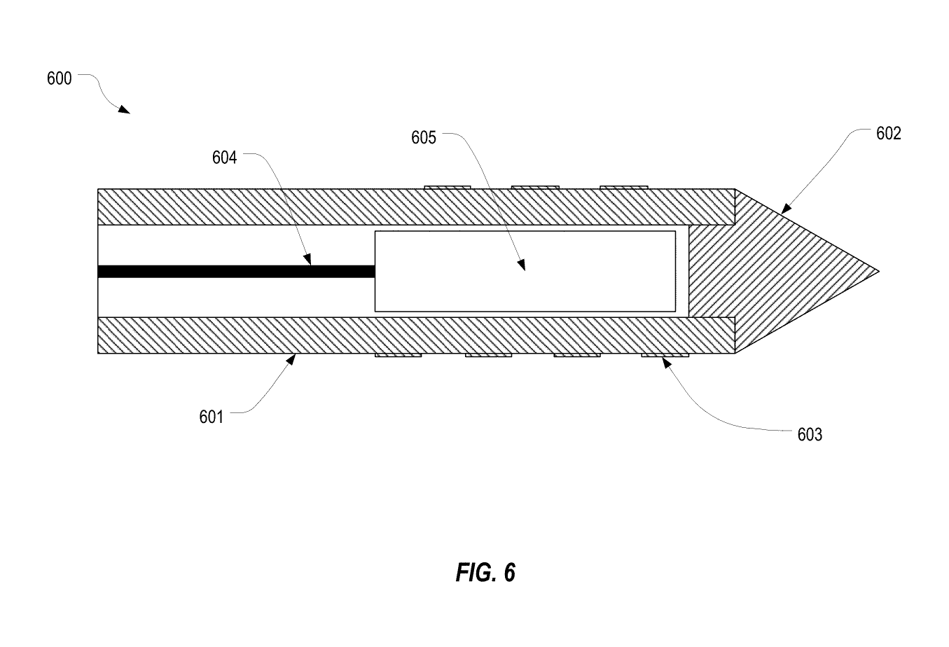

The present invention also encompasses embodiments where a trace is formed on an outer surface of a metalized ceramic component but is not directly connected to a conductor. FIG. 6, for example, provides a cross-sectional view of an ablation probe 600 that includes a shaft 601, a tip 602 (which may be the same component as shaft 601), a conductor 604, and an internal antenna element 605. A trace 603 may be formed on an outer surface of shaft 603 but may not be directly connected to conductor 604 or antenna element 605. In such cases, trace 603 may function to alter the radiation pattern of antenna element 605. Antenna element 605 is shown as a box to represent that any suitable antenna configuration can be used. For example, antenna element 605 may be implemented using traces that are formed on the inner surface of the ceramic shaft 601.

In some embodiments, one or more coatings can be applied to an outer surface of a metalized ceramic component. For example, a coating can be applied overtop traces 303 on the outer surface of shaft 301. Using a coating can isolate the traces from a patient's tissue, protect the traces from decomposition (e.g. via oxidation), and provide a smooth surface. In some embodiments, this coating can be comprised of glass which may be preferred due to its dielectric properties which helps radio frequency waves emitted from the traces transition into surrounding tissue.

In some embodiments, a material that provides a non-stick surface may be preferred for the coating. For example, a coating can be formed of Polytetrafluoroethylene (PTFE), glass, or diamond like carbon to prevent ablated tissue from sticking to the outer surface of the coating component and to potentially increase its lubricity. In some embodiments, a glass coating can be employed with an additional PTFE coating overtop the glass. In this way, the benefits of a glass coating can be obtained while also having a non-stick PTFE surface. Other combinations of coatings may also be applied to all or a portion of an outer surface of a probe.

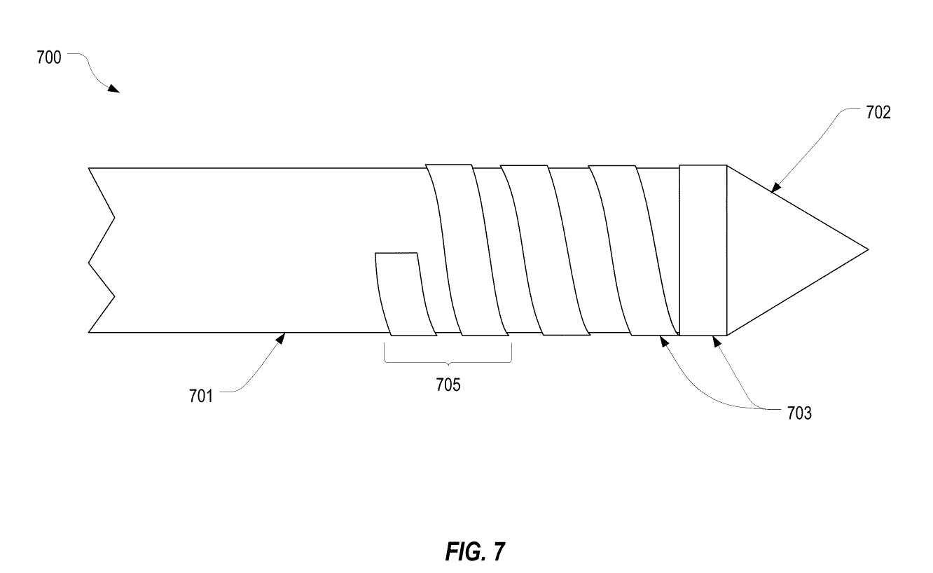

FIG. 7 illustrates an embodiment of a probe 700 having a trace 703 with a varying pitch. Probe 700 includes a shaft 701 and a tip 702 which may be separate components or the same component. One or both of shaft 701 and tip 702 may be comprised of ceramic. Trace 703 forms a helical pattern that extends from tip 702 proximally along an outer surface of shaft 701. The pitch of trace 703 is smaller at a proximal end 705 of the trace such that the windings of trace 703 are closer together at the proximal end. Other variations in the pitch of a trace may also be employed. Also, one or more traces with varying pitches can be used in any of the above described embodiments. The trace patterns may also be configured in a non-helical pattern.

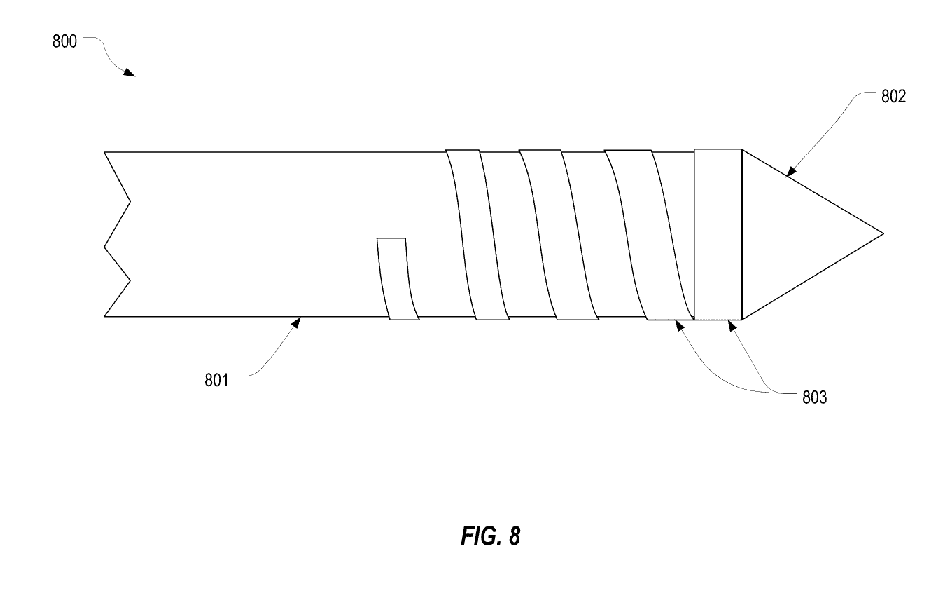

FIG. 8 illustrates an embodiment of a probe 800 having a trace 803 with a varying width. Probe 800 includes a shaft 801 and a tip 802 which may be separate components or the same component. One or both of shaft 801 and tip 802 may be comprised of ceramic. Trace 803 for a helical pattern that extends form tip 802 proximally along an outer surface of shaft 801. As shown, the width of trace 803 narrows at a proximal end of the trace. Other variations in the width of a trace may also be employed. Also, one or more traces with varying widths can be used in any of the above described embodiments.

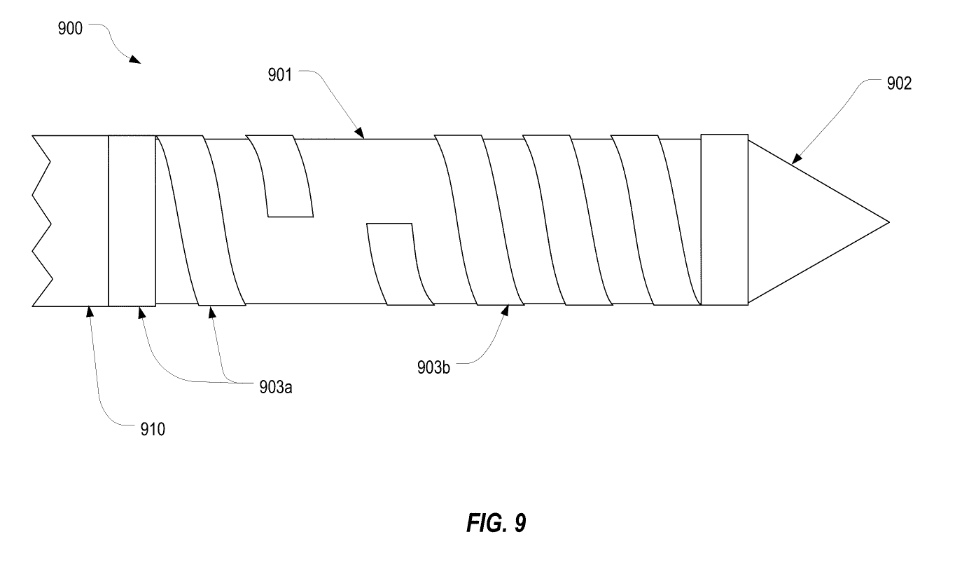

FIG. 9 illustrates an embodiment of a probe 900 having multiple traces 903a, 903b. Probe 900 includes a proximal shaft 910, a shaft 901 positioned distally to proximal shaft 910, and a tip 902. Each of proximal shaft 910, shaft 901, and tip 902 may be separate components, or two or more may be a single component. For example, shaft 901 and tip 902 may be formed of a single ceramic component. Similarly, proximal shaft 910, shaft 901, and tip 902 may be formed of a single ceramic component. Trace 903a extends distally along shaft 901, while trace 903b extends proximally towards trace 903a. In some embodiments, trace 903a and trace 903b may connect. Trace 903a may be connected to an outer conductor (not shown) which forms a ground, while trace 903b may be connected to an inner conductor. One or both of traces 903a and 903b may include variations in pitch, width, pattern, or another parameter as described above.

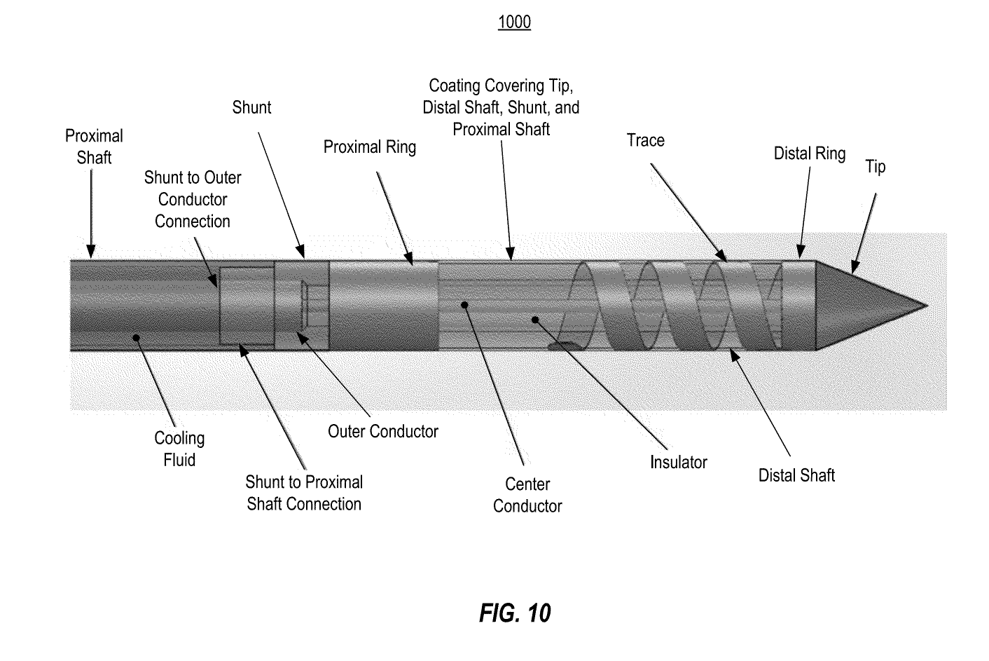

FIG. 10 illustrates another example probe 1000 that includes one or more ceramic components. In typical implementations, the distal shaft of probe 1000 can be formed of ceramic. However, the tip and/or proximal shaft may also be formed of ceramic. Probe 1000 includes a trace that extends around the distal shaft 3.5 times in a helical pattern. The trace may be connected to a center conductor via the distal ring to form the microwave element. The distal ring may form a connection with the center conductor in any of the ways described above including via a conductive tip, via traces formed on a non-conductive tip, or via a direct connection with the center conductor. One or more outer coatings (e.g. glass and/or PTFE) cover the entire probe (i.e. the tip, distal shaft, shunt, and proximal shaft). A shunt (which, in some of the above described embodiments, may be similar to proximal shafts 410, 510, and 910) extends into both the proximal shaft and the distal shaft and functions to form an electrical connection between the outer conductor and the proximal ring, and to form a thermal connection between the tip and the cooling fluid contained within the proximal shaft.

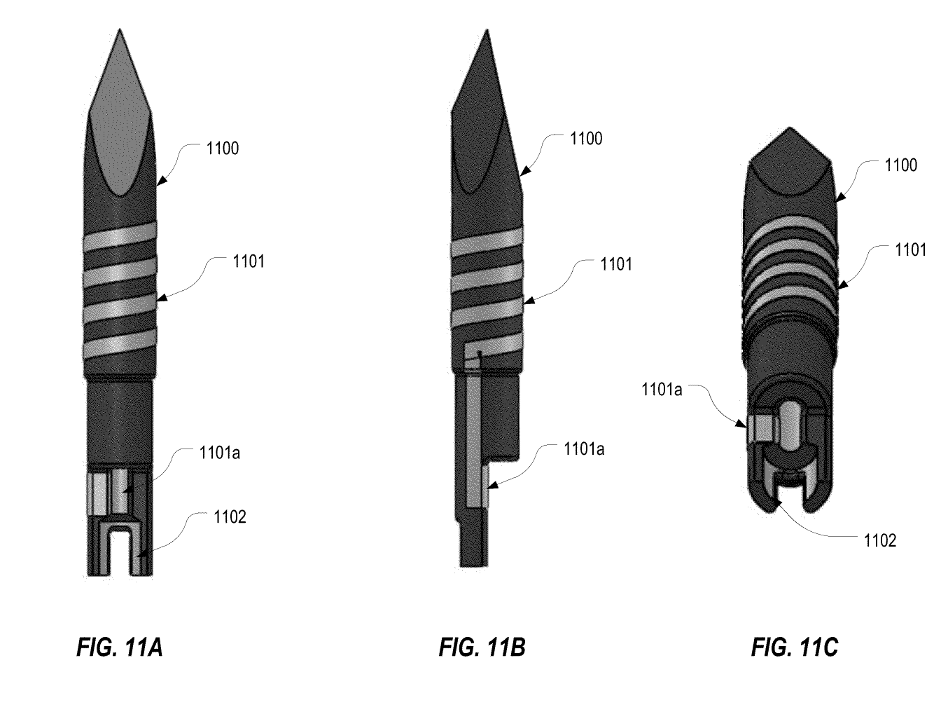

FIGS. 11A-11C illustrate different views of a probe 1100 that is comprised of ceramic. A distally extending trace 1101 is formed on the outer surface of probe 1100. Trace 1101 includes an extension 1101a that extends onto an inner surface of probe 1100 for connection with an inner conductor (not shown). Probe 1100 can also include a metalized portion 1102 for forming an electrical connection with an outer conductor (not shown). Although not shown, probe 1100 may also include a proximally extending trace which may function as a ground trace.

In some embodiments, an insulative coating (not shown) can be applied on probe 1100 prior to forming trace 1101. One or more outer coatings (e.g., glass and/or PTFE) may also be applied overtop trace 1101 after trace 1101 is formed. In some embodiments, probe 1100 can be configured to be inserted within a shaft (not shown) within which the inner and outer conductors are contained. In some embodiments, probe 1100 may have a blunt or rounded tip for fitting inside an external shaft.

In any of the above described embodiments, the tip can be formed of more than one piece. For example, in some embodiments, a tip may comprise an inner metallic piece and an outer non-conductive piece which may be formed of ceramic. The inner metallic piece may form a connection between a conductor and a trace formed on the outer non-conductive piece and/or on a shaft to which the tip is connected.

In some embodiments, one or more inner coatings may be applied to the proximal and/or distal ends of a metalized ceramic component. For example, one or more inner coatings may be applied within the proximal and distal ends of shafts 301, 401, or 501 such as overtop traces 303, 403, 503a, or 503b. Inner coatings can be applied to enhance the connection and/or increase the conductivity between connecting components such as between a distal ring and a tip. One or more inner coatings may also be applied other components such as over traces 305, 405, 505a, or 505b.

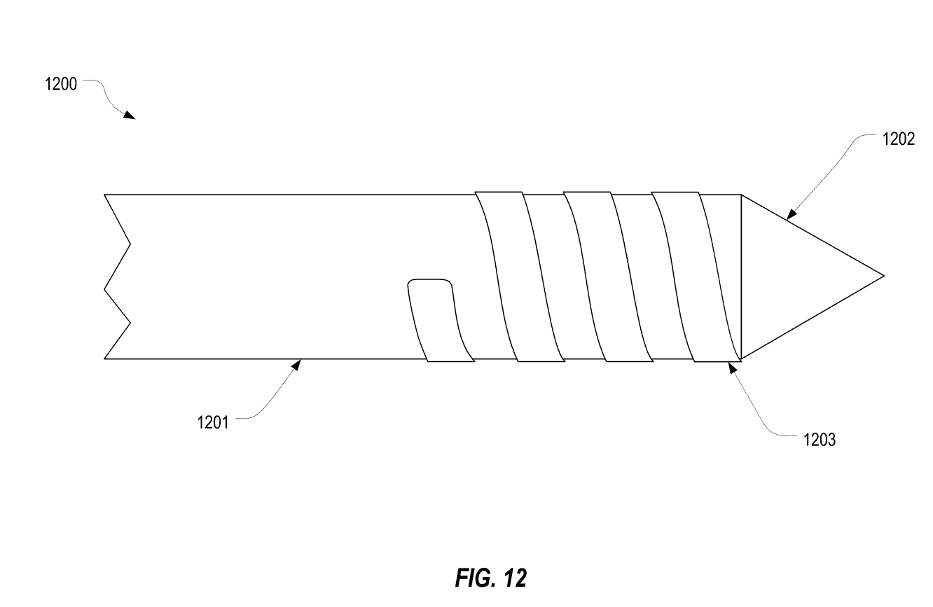

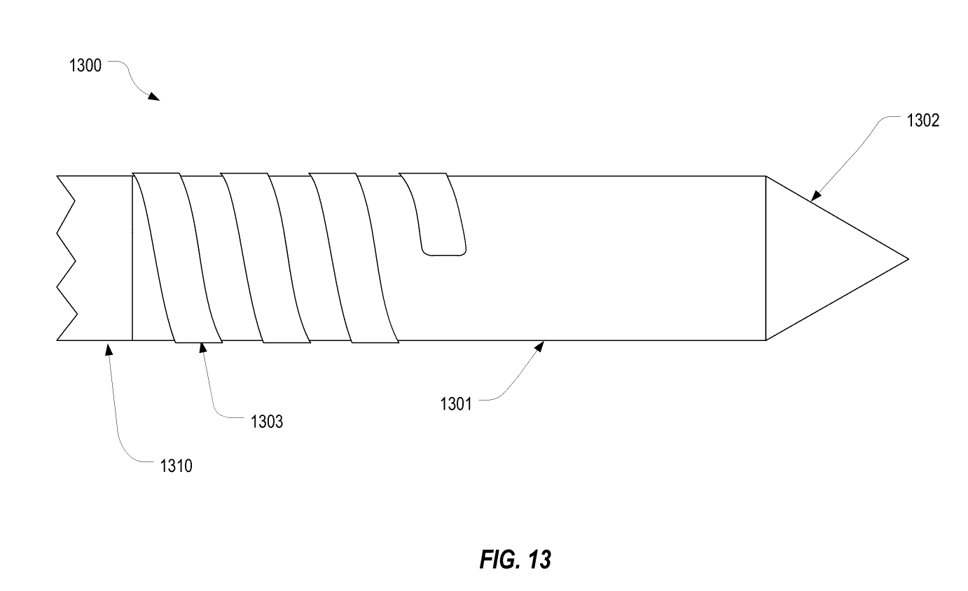

In many of the above described embodiments included those shown in FIGS. 3-5 and 7-10, a trace on the external surface of the component is shown as including a ring at the proximal or distal end of the component such as the proximal and distal rings labeled in FIG. 10. Such a ring, however, is not required. FIGS. 12 and 13 each illustrate an embodiment where a trace extends directly from an end of the shaft and does not include a ring.

FIG. 12 illustrates a probe 1200 similar to probe 300 that includes a shaft 1201, a tip 1202, and a trace 1203. In contrast to trace 303 on probe 300, trace 1203 does not initially extend around shaft 1201 to form a ring, but immediately commences a helical pattern. Of course, trace 1203 may form another non-helical pattern in any of the manners described above.

FIG. 13 illustrates a probe similar to probe 400 that includes a shaft 1301, a tip 1302, a proximal shaft 1310, and a trace 1303. As in FIG. 12, trace 1303 does not form a ring, but immediately commences a helical pattern. Trace 1303 may also form another non-helical pattern. In some embodiments, a trace similar to trace 1203 could be included on shaft 1301, or a trace similar to trace 1303 could be included on shaft 1201.

In summary, the present invention is generally directed to a probe for use in MWA procedures that includes one or more metalized ceramic components. A ceramic component may be metallized to form an antenna, ground plane, or other conductive trace for carrying or emitting microwave energy. Ceramic components provide high heat tolerance thereby allowing a probe containing such ceramic components to be effectively operated at levels that produce large amounts of heat.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed:

1. An ablation probe comprising a shaft formed of a metalized ceramic.

2. The ablation probe of claim 1, wherein the shaft is metalized by forming one or more traces along a surface of the shaft.

3. The ablation probe of claim 2, wherein the one or more traces extend along an outer surface of the shaft.

4. The ablation probe of claim 3, wherein the one or more traces also extend along an inner surface of the shaft.

5. The ablation probe of claim 2, wherein the one or more traces extend along a surface of the shaft that is in contact with a tip or a proximal shaft of the ablation probe.

6. The ablation probe of claim 5, wherein the tip or proximal shaft is formed of a dielectric material, a metalized ceramic, or of a conductive material.

7. The ablation probe of claim 6, wherein the tip or proximal shaft connects the one or more traces to a conductor that extends within the shaft.

9. The ablation probe of claim 2, further comprising:
one or more coatings on the surface of the shaft covering at least a portion of the one or more traces.

10. The ablation probe of claim 2, wherein at least one of the one or more traces transmits electromagnetic waves.

11. The ablation probe of claim 2, wherein the one or more traces comprise a distal trace and a proximal trace, the distal trace being electrically connected to a first conductor and the proximal trace being electrically connected to a second conductor.

12. The ablation probe of claim 11, wherein the distal trace is electrically connected to the proximal trace.

13. The ablation probe of claim 2, further comprising a tip formed of metalized ceramic on which a tip trace is formed, the tip trace contacting at least one of the one or more traces on the shaft.

14. The ablation probe of claim 1, wherein the shaft comprises a tip.

15. The ablation probe of claim 2, wherein at least one of the one or more traces has a varied dimension or pattern.

16. An ablation probe comprising:
a shaft that is formed of ceramic and that includes one or more metal traces formed on a surface of the shaft; and
a tip configured for insertion into a patient to perform an ablation procedure.

17. The ablation probe of claim 16, wherein the tip is formed of a conductive material, at least one of the one or more metal traces being in contact with the tip for receiving electrical energy that is conducted through the tip.

18. The ablation probe of claim 17, wherein at least one of the one or more metal traces is formed on an outer surface of the shaft.

19. The ablation probe of claim 16, wherein the tip is formed of ceramic.

20. The ablation probe of claim 19, wherein the tip includes one or more metal traces on a surface of the tip, the one or more traces on the surface of the tip being connected to the one or more metal traces on the surface of the shaft.

21. The ablation probe of claim 16, wherein the shaft and the tip are a single component.

22. The ablation probe of claim 16, wherein at least one of the one or more metal traces has a varied dimension or pattern.

23. An ablation probe comprising:
a body that is formed of ceramic; and
one or more traces formed on a surface of the body.

24. The ablation probe of claim 23, wherein the one or more traces form an antenna.

25. The ablation probe of claim 23, wherein the body includes a tip.

26. The ablation probe of claim 23, wherein the one or more traces are formed on one or both of an outer surface and an inner surface of the body.

27. The ablation probe of claim 23, wherein at least one of the one or more traces has a varied dimension or pattern.
